(19) United States
(12) Patent Application Publication
(10) Pub. No.: US 2005/0277149 A1
(43) Pub. Date: Dec. 15, 2005

(54) Pharmaceutically tractable secondary drug targets, methods of identification and their use in the creation of small molecule therapeutics

(75) Inventors: Stephen Friend, Seattle, WA (US); Leland Hartwell, Seattle, WA (US)

Correspondence Address: MCDERMOTT WILL & EMERY LLP, 600 13TH STREET, N.W., WASHINGTON, DC, 20005-3096, US

(73) Assignee: Fred Hutchinson Cancer Research Center, Seattle, WA (US)

(21) Appl. No.: 11/150,435

(22) Filed: Jun. 13, 2005

Related U.S. Application Data
(63) Continuation of application No. 09/847,588, filed on May 3, 2001, now abandoned.

(51) Int. Cl. C12Q 1/68; C12N 15/85

(52) U.S. Cl. 435/6; 435/455

(57) ABSTRACT

The present invention relates to a method of identifying one or more secondary drug targets and their use in the identification of drug or drug candidates, particularly for the treatment of cancer. The yeast-based synthetic lethal screens were used to functionally identify and validate new gene targets to kill tumor cells with defects in cell cycle checkpoints and damage response pathways. These newly identified gene targets can be used to develop new cancer chemotherapeutics.

### CROSS REFERENCE TO RELATED APPLICATIONS

The present application claims the benefit of the priority dates of provisional patent application Nos. 60/064,657 and 60/080,471 filed Nov. 6, 1997 and Apr. 2, 1998, respectively. The complete disclosures of these earlier filed applications are incorporated by reference herein.

### FIELD OF THE INVENTION

The present invention relates to the use of synthetic lethal screens to identify secondary drug targets in mammalian tumor cells. The secondary drug targets, once identified, can be used to screen compounds that exhibit antitumor activity.

### BACKGROUND OF THE INVENTION

Anti-cancer drug discovery is now driven by the numerous molecular alterations identified in tumor cells over the past decade. To exploit these alterations, it is necessary to understand how they define a molecular context that allows increased sensitivity to particular compounds. Traditional genetic approaches together with the new wealth of genomic information in both human and model organisms open up strategies by which drugs can be profiled for their ability to selectively kill cells in a molecular context matching those found in tumors. Similarly, it may be possible to identify and validate new targets for drugs that would selectively kill tumor cells with a particular molecular context.

The recent remarkable progress in identifying molecular alterations in human tumors has unfortunately not been paralleled in the field of anti-cancer drug discovery. The shortage of effective anti-cancer drugs is due in part to the fundamental difficulties associated with the development of any safe and effective drug. For example, it remains a formidable task to design small molecules that alter the function of macromolecules with both sensitivity and specificity (for example, an enzyme with a small active site). It is even more difficult to inhibit protein-protein interactions mediated over a large surface, or to restore function to a defective protein (such as an inactive tumor suppressor gene). Even when successful, massive efforts are required—often measured in years to decades—from dozens of chemists, biochemists and toxicologists.

There are also many difficulties specific to anti-cancer drug discovery programs. An effective chemotherapeutic must selectively kill tumor cells. Most anti-cancer drugs have been discovered by serendipity and the molecular alterations that provide selective tumor cell killing are unknown. Even understanding the detailed molecular mechanism by which a drug acts often provides little insight into why the treated tumor cell dies. For example, it is not understood why cisplatin, a DNA cross linking agent, is an effective chemotherapeutic for most germline testicular tumors. See, B. Koberle et al., Int. J. Cancer 70, 551 (1997); R. L. Comis, Semin. Oncol. 21, 109 (1994).

Novel, more effective anti-cancer drugs are likely to require both an awareness of sensitizing molecular contexts (that is, how the tumor cell differs genetically from the normal cell) and which patients have these sensitizing defects.

As the genome sequencing projects finish sequencing the genomes of organisms such as the yeast (S. cerevisiae) (Hahn, H. et al., in Cell (1996) 85: 841-51), and make significant progress towards completing the sequence of others such as fruit flies (D. melanogaster), and worms (C. elegans), homologs of mammalian genes become quite plentiful. This has been helpful in identifying potential “candidate tumor suppressor genes” such as the gene altered in basal cell carcinomas BCC, which in part was cloned through knowledge of an interesting gene called “patched” in flies (Johnson, R. L. et al., in Science (1996) 272: 1668-71).

Information regarding the homologs in diverse species amenable to genetic screens has also been helpful in many instances for gaining a better idea of the function of their mammalian cousins. The function of the bcl-2 gene, found altered in human tumors, was pivotally assigned through studies on its homolog in worms, ced-9 (Rijsewijk, F. et al., in Cell (1987) 50: 649-57). Combined with a plethora of data gathered by many molecular and genetic studies, the list of molecular alterations in tumors has grown enormously within the last decade. One would think that this list of oncogenes, tumor suppressor genes and genes controlling genetic stability would be a rich source of targets for the development of anti-cancer agents.

Unfortunately, the vast majority of these genes do not present pharmaceutically tractable targets for the creation of small molecule therapeutics.

The most common molecular targets, which have proven useful in the identification of small molecule drugs, are enzymes, receptor-ligand pairs, and occasionally specific protein-protein interactions. Selective inhibitors of these types of molecular processes can readily be found that block the biochemical reactions carried out by these molecules (Gibbs, J. B. and Oliff, A., in Cell (1994) 79: 193-8). However, many of the genetic abnormalities found in human cancers represent loss of function mutations that eliminate or severely reduce the biochemical activities governed by these proteins. Since these molecules have already lost their normal biochemical activities, blockade of their physiological functions by drug inhibitors offers no therapeutic benefit. Thus, the list of potential cancer drug targets is much smaller than the long list of genes that are altered in human tumors.

An example of a potentially revealing genetic screen, performed in fruit flies, involves using combinations of partial mutants that together are synthetically lethal. Working with a homolog of the growth factor TGF-beta called decapentaplegia (dpp), Sekelsky and co-workers found synthetic lethality between mutations in dpp and a novel gene they coined Mad (for mothers against dpp) (Sekelsky, J. J. et al., in Genetics (1995) 139: 1347-58). Again, an unexpected gene can be identified, which might allow selective killing of tumor cells when the primary defect is found distinctly in tumor cells.

It has now been discovered by the present invention that yeast genetics can be used to streamline anti-cancer drug discovery. In particular, in an effort to identify genetically validated and pharmacologically feasible targets, certain model organisms have been revisited for novel ways of applying classic genetic strategies, this time to help in the search for alternate or “secondary” drug targets.

Briefly, geneticists studying model organisms such as fruit flies, worms and yeast have long recognized that identification of a gene responsible for a given phenotype is only the first step in the process of understanding its function. Empowered by the ability to search through large numbers of organisms that have been mutated to generate new phenotypes, geneticists have developed methods for identifying genes whose protein products profoundly influence other genes. Specifically, geneticists have perfected assays, called “synthetic lethal screens.” These screens begin with a particular genetic context, e.g., inactivation of a primary gene that may have little effect on the viability of the whole organism, and then mutate all of the organism's remaining genes among a large sampling (e.g., thousands to hundreds of thousands) of the organism's offspring. The end product of these assays is the identification of a secondary gene that, if inactivated, will selectively kill only those cells that also contain the primary defect. For more information on synthetic lethal gene screening see, for example, Doye et al in Trends in Genet. (1995) 11:235; Koshland, J. C. et al. in Cell (1985) 40:393; Hieter, C. et al. in Cell (1985) 40: 381; Riles, L. et al. in Genetics (1988) 118: 601; Bender, A. et al. in Mol. Cell. Biol. (1991) 11:1295; and Kaiser, C. A. et al. in Cell (1990) 61: 723.

Anticancer drug discovery is now driven by the numerous molecular alterations identified in tumor cells over the past decade. To exploit these alterations, it is necessary to define a molecular content that allows increased sensitivity to particular compounds. Traditional genetic approaches together with the new wealth of genomic information in both human and model organisms open up strategies by which drugs can be profiled for their ability to selectively kill cells in a molecular context matching those found in tumors. Similarly, it may be possible to identify and validate new targets for drugs that would selectively kill tumor cells with a particular molecular context. This invention explores the avenues in which molecular genetics can be used to streamline anti-cancer drug discovery. As discussed further below, the present invention demonstrates the desirability of performing synthetic lethal genetic screens for the identification of unexpected drug discovery targets for cancer.

### SUMMARY OF THE INVENTION

The present invention is directed generally to a method of identifying one or more secondary drug targets comprising the steps of providing a cell having at least one primary gene defect in the genome; effecting one or more mutations in the genome of the cell, at one or more secondary sites; selecting at least one secondary site mutation that proves lethal to the cell; and determining the gene product of the lethal secondary site to provide a secondary drug target.

In a particular embodiment of the invention, the primary gene defect is preferably one found in or associated with a mammalian tumor, more preferably a human tumor. Alternatively, the primary gene defect in the cell provided by the instant method is analogous or homologous to a defect found in or associated with a mammalian or human tumor. By “homologous” is meant a direct relationship among a “family” of genes in which certain sequences or domains are strongly conserved among the members of the family. For instance, the yeast mec1 gene is homologous to mammalian genes encoding AT-related kinase. On the other hand, “analogous” genes may serve similar or “analogous” functions, but they are not directly related (i.e., sequences are not conserved among analogous genes). There may be human analogs of yeast slm1 (synthetic lethal with mec-1) and MBP1 genes.

In the present method, the primary gene defect may result in the alteration, loss, or inhibition of a function, for example, a cellular function. However, the primary gene defect may also result in the enhancement or gain of a function. Certain cyclin-dependent protein kinases, for example, can be activated by a primary gene defect that gives rise to cyclin overexpression. In contrast, the expression of p16 or p27 can inhibit the kinase activity. Hence, a loss of p16 or p27 related function produces a hyperactive kinase.

Generally, the functions affected can vary widely. The affected functions may include, but are not limited to, the suppression of tumor growth, DNA damage checkpoint, DNA mismatch repair, nucleotide excision repair, O6-methylguanine reversal, double-strand break repair, DNA helicase function, signaling, cell cycle control, or apoptosis. In a particular embodiment of the invention, the signaling function includes, but is not limited to, signal transduction, tissue growth factor signaling, autocrine loop signaling, or paracrine loop signaling. In one method, the sought after primary gene defect may include a defect in a mammalian gene coding for p16, p53, ATM, MSH2, MLH1, XP-A, XP-B, MGMT, BRCA2, BRCA1, BLM, RAS, NF1, MYC, PTH, Cyclin D, Cyclin E, p27kip1, Rb, or BCL-2. Such defects may be effectively modeled by primary gene defects in other organisms, including a defect in the gene coding for RAD9Sc, rad1+Sp, MEC1Sc, TEL1Sc, rad3+Sp, mei-41Dm, MSH2Sc, MLH1Sc, RAD14Sc, RAD25Sc, MGT1Sc, RAD51Sc, RAD54Sc, SGS1Sc, rqh1+Sp, dRASDm, RASCe, RAS1Sc, RAS2Sc, let-60Ce, IRA1Sc, IRA2Sc, dMycDm, patchedDm, CLN1Sc, CLN2Sc, Cyclin DDm, Cyclin EDm, SIC1 Sc, RbfDm, or ced-9Ce.

By the methods of the present invention, it has been found that certain secondary site mutations can be effected, which may turn out to be lethal to the cell harboring the primary gene defect. Such secondary site mutations may be effected, for example, within a gene selected from the group consisting of cdc9, cdc2, a gene encoding a gene product exhibiting polymerase δ exonuclease function, a gene encoding a gene product exhibiting polymerase ε exonuclease function, a gene encoding a ribonucleotide reductase, mec1, rad53 like gene, cdc53, cdc34, cdc14, cdc15, a gene encoding NUP170, dbf2, a gene encoding CLN2, rad3, rad9, rad27, cdc8, a gene encoding Mlu1-box binding factor, slm1, a gene encoding MBF, a gene encoding PCNA, or a gene encoding replication fork protein.

Most preferably, the secondary site mutation is effected within a gene having a mammalian analog or homolog. In a preferred embodiment of the invention, the homologous mammalian gene is selected from the group consisting of a gene encoding a DNA ligase I, a gene encoding a DNA polymerase, a gene encoding a ribonucleotide reductase, a gene encoding a FEN-1, a gene encoding Cyclin D, a gene encoding Cyclin E, an AT-related gene, a gene encoding NUP155, or a gene encoding an isozyme.

A further object of the invention includes the identification of drug or drug candidates. Hence, after the secondary drug target is elucidated, the secondary drug target can be used to screen for a drug or drug candidate that can potentially interact with the secondary drug target, for example to disable its physiological activity. Accordingly, the present invention may provide a drug or drug candidate that interacts with, binds to, or inhibits a particular gene product. Such gene products may include, but are not limited to, DNA ligase, DNA polymerase, polymerase δ exonuclease function, a gene encoding a gene product exhibiting polymerase ε exonuclease polymerase, ribonucleotide reductase, a subunit of transcriptional activator, a transcription factor, PCNA, a replication fork protein, PIK-related kinase, recombinase, E3 ubiquitin ligase, E2 ubiquitin carrier protein, a protein tyrosine phosphatase, a nuclear pore protein, cyclin, DNA repair exonuclease, thymidylate kinase, gene product of slm1, ribonucleotide reductase, or a transcriptional activator. It is desirable that the drug or drug candidate exhibit the capacity to inhibit or arrest the growth of a human tumor. Most preferably, administration of the drug or drug candidate results in death of the tumor cell, reduction in neoplastic tissue and a cure for the cancer.

Thus, the present invention provides a method of rational antitumor drug design comprising: (i) providing a genetically tractable organism harboring an altered gene that is analogous or homologous to a primary tumor defect, (ii) performing a synthetic lethal screen to identify a secondary target gene, (iii) determining an analogous or homologous secondary target in mammalian cells, and (iv) using the analogous or homologous secondary target to screen for a drug or drug candidate having antitumor activity. Preferably, the drug or drug candidate comprises a small molecule. The activity of the small molecule can subsequently and optionally be optimized by conventional medicinal and/or synthetic chemistry methods.

In a preferred method of the invention, additional steps are performed comprising validating the synthetic lethality of the analogous or homologous secondary target in a mammalian tumor cell relative to a mammalian non-tumor cell. Most preferably, the resulting drug or drug candidate will prove to be selective, even specific, for tumor cells. In this manner, the invention contemplates a method of treating a cancer comprising administering to a cancer patient an effective amount of an anticancer agent, which anticancer agent interacts with, binds to, or inhibits a gene product of a secondary target gene present in a mammalian tumor cell. The secondary target gene is identified by performing a synthetic lethal screen. Use of the gene product of the secondary target gene to screen a library of compounds provides the identification of a desired anticancer agent effective against the mammalian, preferably human, tumor cell.

The present invention provides a pharmaceutical composition comprising an effective amount of an agent derived from the gene product of a lethal secondary site mutation, the expression of which proves lethal to a cell having at least one primary gene defect, and a pharmaceutically acceptable carrier or diluent. The agent of the pharmaceutical composition comprises the gene product of interest, active fragments thereof, derivatives or analogs thereof, or small molecule or peptide mimetics thereof. In another embodiment of the invention, a pharmaceutical composition is provided which comprises an effective amount of an agent and a pharmaceutically acceptable carrier or diluent, the agent capable of inhibiting either the expression of a synthetic lethal gene or the activity of the gene product of a synthetic lethal gene that is found in a cell having at least one primary gene defect.

A further object of this invention includes a pharmaceutical composition comprising a drug, in a pharmaceutically acceptable carrier or diluent, which drug selectively interacts with the production of at least one gene product in a cell population that contains at least one primary gene defect, wherein the exposure of the cell population to the drug arrests cell division selectively in the cell population. Such gene products are encoded or regulated by a human gene analogous or homologous to a yeast gene, wherein the yeast gene comprising, cdc9, cdc2, a gene product exhibiting polymerase 8 exonuclease function, a gene product exhibiting polymerase ε exonuclease function, a ribonucleotide reductase, mec1, rad53 like gene, cdc53, cdc34, cdc14, cdc15, NUP170, dbf2, CLN2, rad3, rad9, rad27, cdc8, Mlu1-box binding factor, slm1, MBF, PCNA, a gene encoding replication fork protein. RNR1, RNR2, RNR4, CDC21, SHM2, PR11, CDC17, MBP1, slm 2, slm 3, and slm 4.

Yet a further object of this invention is to provide a pharmaceutical composition and method effective against cancer wherein the active ingredient of the composition is an agent derived from the gene product of a lethal secondary site mutation in a human gene, which agent is selected from the group consisting of inhibitors of protein-coupled receptors, agonists, antagonists, growth hormones, ligands, antibodies, wherein the human gene comprises a gene encoding a DNA ligase I, a gene encoding a DNA polymerase, a gene encoding a ribonucleotide reductase, a gene encoding a FEN-1, a gene encoding Cyclin D, a gene encoding Cyclin E, an AT-related gene, a gene encoding NUP155, or a gene encoding an isozyme.

Hence, a method of treating cancer is contemplated, which includes the administration of effective amounts of the pharmaceutical compositions of the present invention. In a particular embodiment, a method of treating cancer cells having abnormal accumulation of a human G1/S Cyclin is disclosed, which method comprises administering a pharmaceutical composition comprising an effective amount of an agent and a pharmaceutically acceptable carrier or diluent, the agent capable of inhibiting either the expression of a synthetic lethal gene or the activity of the gene product of a synthetic lethal gene that is found in a cell having at least one primary gene defect, wherein said gene product is selected from (or wherein said synthetic lethal gene codes for) a human isozyme of cdc34, a human isozyme of cdc53, a human isozyme of skp1, a human isozyme of cdc14 and NUP155. In a preferred method, either the gene product comprises ATR or the synthetic lethal gene codes for ATR. Specifically the method of the invention uses ATR-dk as a synthetic lethal gene.

These and other objects of the present invention are evident from the disclosure provided herewith.

### BRIEF DESCRIPTION OF THE FIGURES

FIG. 1: Outlines a synthetic lethal screening strategy for identifying secondary targets.

FIG. 2: Cell Cycle/DNA Damage Response Pathways. Schematic showing DNA damage on PIK-domain proteins (ATM, ATR, DNA-PK)

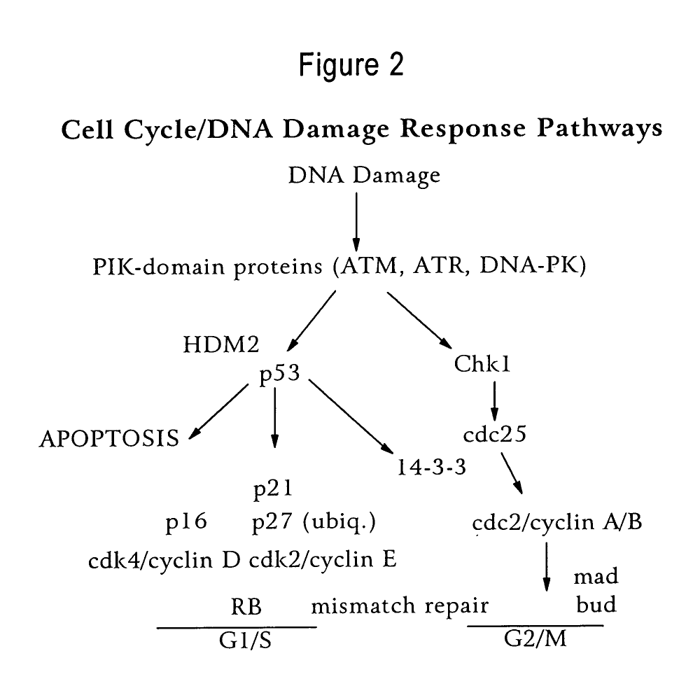

FIG. 3: Mammalian Cell Evaluation Of ATR as a Target.

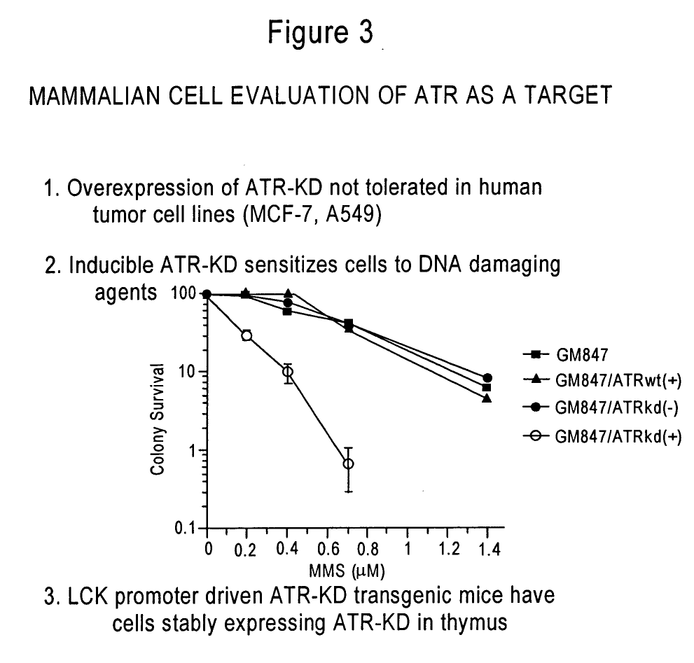

FIG. 4: Synthetic Lethality: Schematic Showing The Use of Primary Defect In Combination To a Secondary Defect on Normal and Tumor Cells.

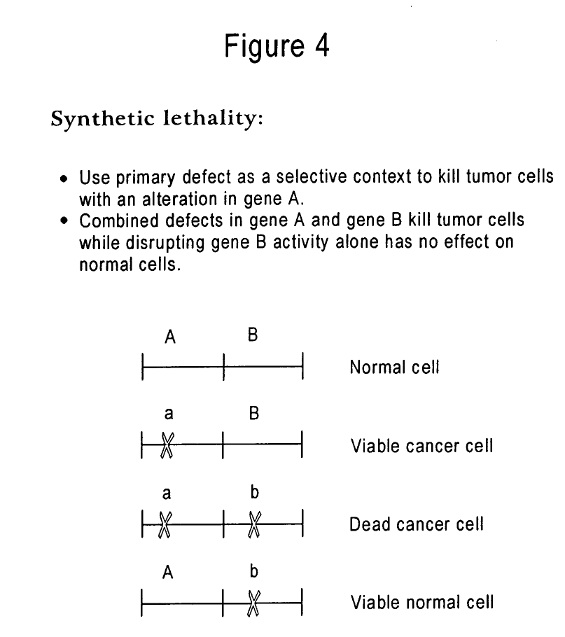

FIG. 5: Human Genes Altered In Tumors and Their Relatives In Model Genetic Systems.

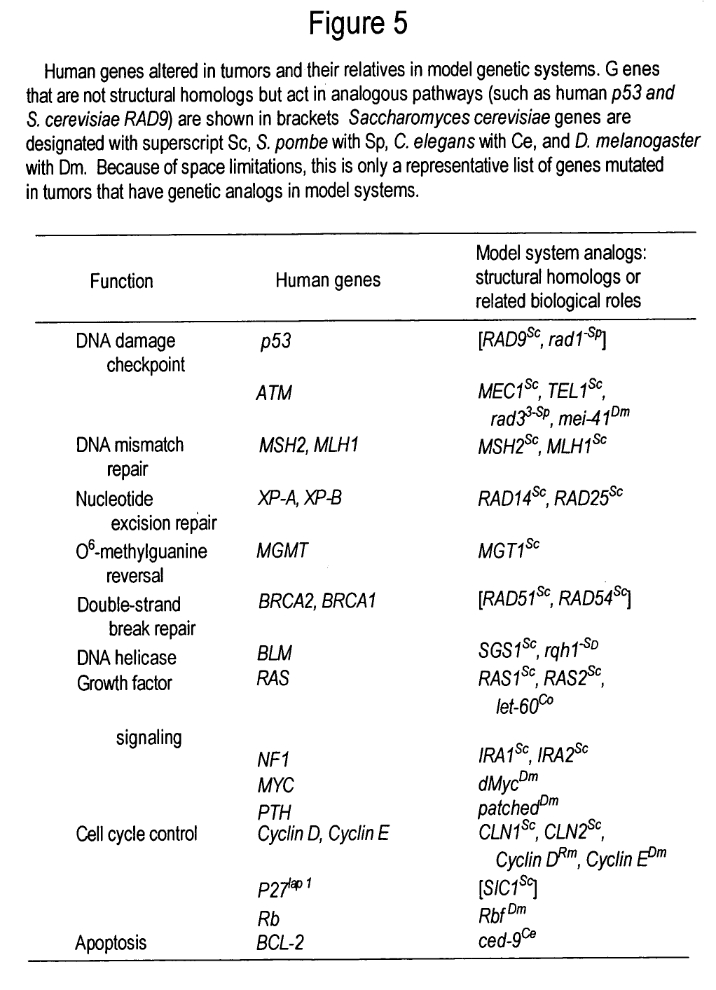

FIG. 6: Cell cycle/DNA damage response pathways. DNA damage of mammalian cells leads to activation of protein kinases such as ATR. These kinases can then influence a number of pathways that control the cell's decision to arrest in G1/S or G2/M phases of the cell cycle or alternatively to undergo apoptosis (cell death). These pathways are highly involved in cancer as the genes, which are underlined, are often found defective in human tumors. These pathways provide a context for interpreting the yeast synthetic lethal results. As an example, kinase defective mec1 (the yeast homologue of mammalian ATR) is synthetic lethal with deregulated cyclin expression, a downstream component of the ATR pathway.

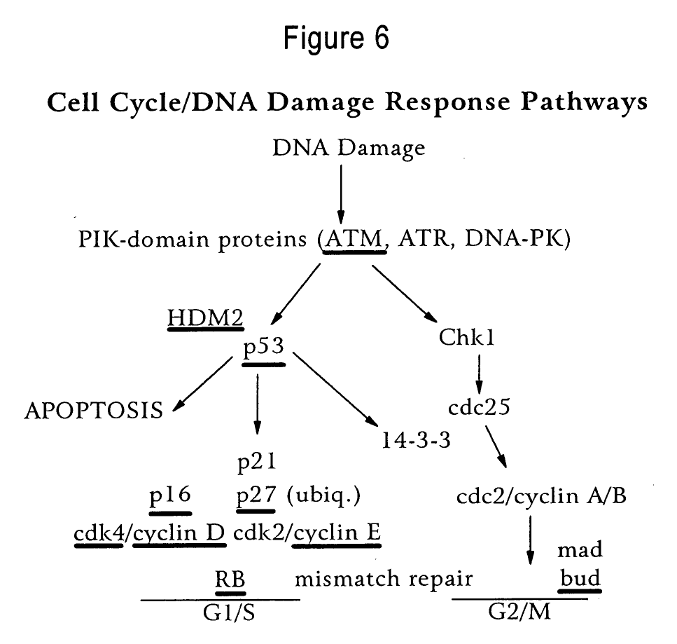

FIG. 7. Mammalian Cell Evaluation of ATR As a Target. ATRkd expression renders cells sensitive to several DNA-damaging agents. Clonogenic survival of GM847, GM847/ATR wt, GM847/ATRkd and AT3B1 fibroblasts, in the presence (+) or absence (−) of deoxycycline, is determined after exposure to increasing doses of ionizing radiation (IR), cis-platinum, methyl methanesulfonate (MMS) and UV radiation. Plating efficiency is determined for all cell lines and ranges from 12% to 16%. All measurements are performed in triplicate and consistent results are obtained among experiments. In GM847 and GM847/ATR wt cell lines the clonogenic survival is not affected by the presence or absence of doxycycline.

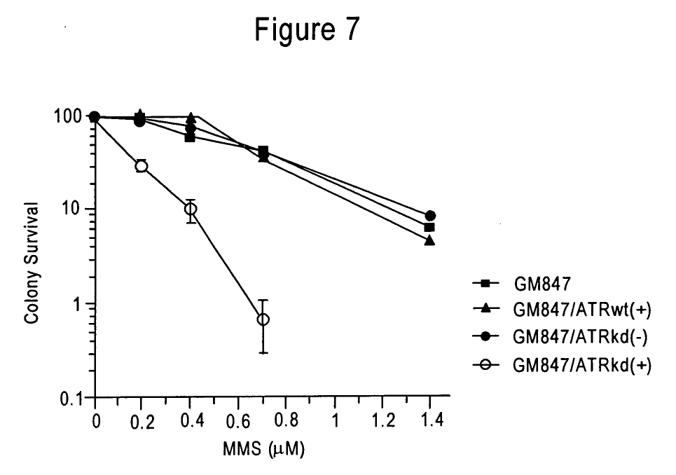

ATR is a phosphatidylinositol kinase-related protein homologous to ataxia telangiectasia mutated (ATM). This protein is important for the survival of human cells following many forms of DNA damage. The results of experiments with ATR target reveal that overexpression of ATRkd is not tolerated in human tumor cell lines MCF-7 and A549. Inducible ATRkd sensitizes cells to DNA damaging agents. Transgenic mice transformed with LCK promoter driven ATRkd, stably express ATRkd in thymus.

### DETAILED DESCRIPTION OF THE PREFERRED EMBODIMENTS

The present invention concerns a method of identifying drugs that selectively inhibit the growth of particular cancer cells as well as methods of using such drugs. The invention features a method of identifying mutant organisms having a secondary site mutation, which is lethal to the cell, and, subsequently, gene products thereof. The disclosed methods are useful for high-throughput screening of genomic or mutant libraries to rapidly identify genes, and corresponding gene products, which are essential for survival. A lethal mutation results in a gene or a protein that is not functional under restrictive conditions (i.e., in a tumor cell). A non-functional gene can have a defect in the promoter resulting in a reduced or abnormal gene expression. A non-functional protein may have a conformational defect causing improper protein folding or abnormal protein degradation. Improper protein folding can result in partial or total failure to fold, to recognize a native substrate, and/or to bind and release the substrate.

Therapeutic agents can be developed from the identification of essential genes of organisms such as bacteria or fungi. For example, a gene selection method could establish that the gene product (i.e., a protein or an RNA molecule) is essential for survival of certain types of cancer cells. Such an identified gene product therefore serves as a novel target for therapeutics, based on a mechanism of action that is likely to be distinct from the mechanisms of existing antitumor drugs. Similarly, distinct from known compounds is a compound that inhibits the function of a gene product identified by the methods disclosed herein, for example, by producing a phenotype or morphology similar to that found in the original mutant strain.

According to one aspect of the invention, a mutant collection is systematically screened to identify genes and preferably gene products, which are targets for drugs. For example, a drug may act as a biocide by binding reversibly, or preferably irreversibly, to the identified gene or gene product target, and thereby impairing its function. Loss of the function (or the synthesis or the complete processing) of the gene product target will result in inhibition of the tumor cell growth, and preferably will result in the death of the tumor cells. This aspect includes a method for identifying anti-cancer agents, including the step of exposing a gene product corresponding to the wildtype sequence of a mutant sequence identified by methods disclosed herein to the test agent; and selecting agents that impair (preferably, selectively) the function of the gene product.

The following definitions are employed herein:

By “genome” is meant the totality of an organism's genetic material, including any chromosomal genes and extrachromosomal genes (including plasmid born genes, cell organelle associated genes, and the like).

By “agent” or “drug” is meant any active agent that has a biological effect on cell growth or cell cycle including, but not limited to, traditional anticancer drugs. The novel drugs identified by the present invention, as capable of selectively inhibiting the growth of particular cancer cells, are molecules having anticancer activity such as tumor necrosis factor and lymphotoxin, proteins encoded by proto-oncogenes and tumor suppressor genes, antibodies or antibody conjugates that target the activated oncogenes in cancer cells, recptor-coupled proteins, or agonists and antagonists of such receptor-coupled proteins.

Transformed phenotype is a phenotype that is not characteristic of a normal (non-cancerous) cell. A transformed phenotype may include loss of contact inhibition, altered morphology and loss of genetic stability. Properties of a transformed phenotype may include, but are not limited to, changes in cell morphology, nuclear structure, cytoskeleton, growth characteristics, cell metabolism, and/or anchorage independence.

By “mutations” is meant any alterations to the genetic material of a cell including any additions, deletions, or substitutions of nucleotide bases relative to the wild type nucleotide base sequence.

Homologous/non-homologous are defined as follows. Two nucleic acid molecules are considered to be homologous if their nucleotide sequences share a similarity of greater than 40%, as determined HASH-coding algorithms (Wilber, W. J. and Lipman, D. J. Proceedings of National Academy of Science U.S.A. 80, 726-730 (1983). Two nucleic acid molecules are considered to be “non-homologous” if their nucleotide sequences share a similarity of less than 40%. Homologous nucleotide sequences are, for example, 41% to 49%, 50% to 59%, 60%, 69%, 70% to 79%, 80% to 89% or 90% to 99% homologous. Homologous genes have a direct relationship among a “family” of genes in which certain sequences or domains are strongly conserved among the members of the family. For instance, the yeast mec1 gene is homologous to mammalian genes encoding AT-related kinase.

Analogous genes as defined herein may serve similar or “analogous” functions, but they are not directly related (i.e., sequences are not conserved among analogous genes). There may be human analogs of yeast slm1, MBP1 and MBF genes.

By “interaction” is meant total arrest, increase, reduction, or abarrent expression in the expression of the gene product or interaction with the biological activity, transport, binding, folding or other post-translational modifications of the gene product.

Abnormalities in components of the cell cycle surveillance system have been identified in human cancers. These abnormalities include alterations in RB/cyclin D and cyclin E/p16 (80-90% of tumors), p53 (50-60% of tumors), and DNA mismatch repair (10-20% of some tumor types such as colon and pancreatic). Often the primary genetic alteration (RB, p16, p53, or mismatch repair) is a loss of function and so a drug discovery program focused on these defects would require restoring the lost function. An alternative approach is to identify which other protein(s) when inhibited selectively kill cells that have the primary defect.

As p53 alterations are common in tumors, and p53 controls the start of DNA synthesis, there are obvious benefits that come from identifying genes that, if inactivated, selectively kill cells that fail to delay initiation of DNA synthesis after damage. Although yeast have no p53 homologs, they do have checkpoint control genes, such as mec3, which arrest cells after DNA damage (Weinert, T. A., in Radiat. Res. (1992) 132: 141-3). Recently, a synthetic lethal screen was set up to find genes synthetically lethal with an unrelated gene, DNA polymerase alpha primase (Longhese, M. P. et al., in Mol. Cell. Biology (1996) 16: 3235-3244). Fortuitously, one of the genes identified in this DNA primase synthetic lethal screen was mec3. Thus, an unsolicited potential therapeutic strategy to kill cells defective in p53 (by targeting DNA polymerase alpha primase) can be stumbled upon by performing synthetic lethal screen in yeast.

The strategy taken by the inventors of the present invention is to identify these “secondary” targets by genetic screens. These genetic screens allow one to find all proteins which if inhibited will selectively kill cells with a specific primary defect. This type of phenomenon, known as synthetic lethality, is without effect in normal cells lacking the primary defect, but in target cells such a combination of primary and secondary defects is lethal. Thus, when the secondary target is pharmacologically inhibited in combination with a specific genetic defect, it confers a lethal phenotype to the tumor cells. Since the nontumor cells would not have the underlying genetic defect, the pharmacological agent would not be toxic to the normal cells. The synthetic lethal screen has the potential to find targets not available to competitors. The secondary targets could be proper targets either as new genes or as known genes but with a new biological use. Since the new target will be identified based on a functional screen, it serves as a biological validation for the new cancer target.

TABLE 1
Rationale for the screens

| Target | Frequency in Cancer | Yeast homologue |
|---|---|---|
| DNA mismatch repair | 15% of colon and endometrial cancers | msh2, m1h1, pms1 |
| PIL-related kinases (DNA | 15% overall, ATM | mec1 |
| damage surveillance) | | |
| Cyclin overexpression | 10-15% overall, cyclin D1 | CLN 1 and 2 |
| | and E - 30-50% of lung, | |
| | colon, and breast | |
| CDK inhibitor | 50% lung, 60% glioma, 47% | sic1 |
| | prostate, 80% pancreas | |
| BRCA ½ | 5-10% breast and ovarian | Rad 51/52 |
| Mitotic apparatus | Colon cancer cell lines | mad ½, bub ½ |
| Telomerase | Essentially 100% | TLC1 |

Many of the mechanisms that control the cell cycle and maintain genetic stability in mammalian cells are conserved in the yeast Saccharomyces cerevisiae. The initial screens in yeast focused on cyclins, G1/S checkpoints, and mismatch repair. Subsequent screens have evaluated a cyclin-dependent kinase inhibitor, a component of the BRCA1 system, and a G2/M checkpoint. A screen for synthetic lethality with elevated telomerase activity has also started. In some cases, yeast do not have homologues of mammalian genes, such as myc. Synthetic lethal screens based on these targets could be done in Drospohilia. Once appropriate targets are identified, they will then be further validated using functional assays in mammalian tumor cells.

More powerful than biochemical screens that test the interaction of previously isolated and fully characterized proteins, synthetic lethal screens identify all other gene products that, if inhibited, will uniquely kill cells whether or not these secondary proteins directly interact with the primary gene product. Thus, the present invention recognizes that synthetic lethal screens permit investigators the luxury of scanning the entire genome of an organism to find the best available secondary target.

After such a target is found in a model organism, the validation of the drug target shifts to an analysis of its role in mammalian cells. Therefore, not unlike the strategies used in the fruit fly to identify the BCC gene, it is possible to use the same model organisms to find secondary targets for the discovery of drugs to treat basal cell carcinomas. Whether performed in yeast, worms, or fruit flies, such genetic screens and whole genome analyses can be harnessed to accelerate the pace of cancer drug discovery.

The use of a genetic approach for drug discovery can potentially improve on current paradigms in two important ways. First, a mutation is a model of an ideal drug. By removing a single gene in a cell or organism one eliminates the function of one and only one protein as though one had a perfect drug for that target. Second, one of the most powerful aspects of carrying out a genetic screen (a search for mutations anywhere in the genome that produce a desired phenotype) is that it allows the organism to tell the observer which functions are the important one. By identifying genes whose mutations produce the desired therapeutic outcome, one will have simultaneously identified and validated appropriate new drug targets. The current state of human cell genetics does not permit one to exploit genetics for drug discovery, so it is necessary to use “model organisms” for which genetic manipulation is facile.

Many of the genes that are frequently altered in tumors have structural or functional homologous in model genetic systems, including the yeasts Saccharomyces cerevisiae and Schizosaccharomyces pombe, the nematode Caenorhabditis elegans, and the fruit fly Drosophila melanogaster. If the potential drug targets are components required for cell division or DNA repair, where there is significant conservation of function between humans and yeast, then yeast would be the organism of choice. Fruit flies and nematodes are also potentially strong models, especially when conservation from human to yeast is weak or when the target components are present only in the multicellular context. Finally, an increasing number of single gene knockouts in murine embryonic stem cells offer opportunities for working with drug targets that are even more closely related to the human organism. See, B. O. Williams and T. Jacks, Curr. Opin. Genet. Dev. 6, 65 (1996).

Identification of drug targets that would achieve a high therapeutic advantage requires knowledge of how the tumor cell differs from the normal cell. Cancer cells are genetically different from their normal cell counterparts, often having undergone at least a half-dozen mutations. See, K. W. Kinzler and B. Vogelstein, Cell 87, 159 (1996); D. N. Louis and J. F. Gusella, Trends Genet. 11, 412 (1995). It is believed that the genetic changes that give rise to the genetic instability of tumor cells may provide the key to tumor cell sensitivity.

Tumor cells universally exhibit genetic instability. Perhaps the best single documentation of this assertion is that many tumor cells from different origins have been examined for their ability to undergo gene amplification and all exhibit high rates of gene amplification in comparison to normal untransformed cells. See, T. D. Tlsty et al., Cold Spring Harbor Symp. Quant. Biol. 58, 645 (1993). Other indications of genetic instability in tumor cells are their frequent karyotypic abnormalities, multipolar mitoses and nucleotide repeat instability. See, B. Vogelstein et al., Science 244, 207 (1989); C. Lengauer, K. W. Kinzler, B. Vogelstein, Nature 386, 623 (1997); L. H. Hartwell and M. B. Kastan, Science 266, 1821 (1994).

Consistent with these ideas is the suggestion that genetic instability reflects a failure in the cell cycle checkpoint function, as has been described in yeast (Hartwell et al., Science, 246, 629-634 (1989)). These checkpoints are pauses that occur at specific points in the cell cycle for purposes of correcting errors, such as in the fidelity of replicated DNA. While mutations in the checkpoint genes could result in a high frequency of mutations that lead to malignant transformation (Hartwell et al., Science, 246, 629-634 (1989)), the activation of an oncogene could compromise checkpoint function anywhere in the cell cycle. For example, the constitutive expression of the mos oncogene throughout the entire cell cycle could override the normal cell cycle program and abrogate normal checkpoint function. This disruption of normal cell cycle function provides an explanation both for the genetic instability of tumor cells and for the greater sensitivity of tumor cells to chemotherapeutic agents compared to non-tumor cells.

The genetic changes underlying this genetic instability have been identified and they fall into three categories: defects in DNA repair pathways (for example, patients with xeroderma pigmentosum (XP) and hereditary non-polyposis colon cancer (HNPCC) show alterations in nucleotide excision repair and DNA mismatch repair, respectively) defects in cell cycle checkpoints and the ATM gene in the hereditary cancer prone syndrome Ataxia telangiectasia, and defects that cause inappropriate transition from the G1 to the S phase of the cell cycle (e.g., RAS activation, MYC activation, or cyclin D amplification). We will use the term “DNA damage response element or pathway” as a general term to cover all three categories.

The reasons for thinking that the genetic changes underlying genetic instability are valuable for drug discovery are as follows. First, since all cancers are genetically unstable, this is a general context in which to consider cancer therapy. Second, genetic instability is probably necessary for the evolution of the cancer cell to a metastatic state. Third, it is well known that defects in many DNA damage response elements resulting in genetic instability also create vulnerability to killing by certain damaging agents. For example, XP mutations cause sensitivity to ultraviolet light, and mutations in ATM and the breast cancer susceptibility gene BRCA2 cause sensitivity to ionizing radiation. While these strategies attempt to turn genetic instability into an asset for therapeutic advantage, the tumor cell heterogeneity that results from this instability could compromise the effectiveness of anti-tumor drugs identified by this or other means.

In the present method, genetic screens are used to identify protein targets that would create therapeutic advantage in a mutant compared to a wild-type by screening for second site mutations that are lethal in the mutant strain but not in the wild type strain (see later discussion of synthetic lethality).

In yeast, knowledge that a topoisomerase poison is more toxic to a yeast cell that is defective in the DNA double-strand break repair pathway is clinically relevant only if an analogous defect occurs in human tumors and this defect determines sensitivity to topoisomerase poisons. In many respects, the most difficult aspect of the genetic approach to drug discovery is the lack of knowledge about mammalian biological pathways.

Many of the genetic alterations frequently found in tumors are loss-of-function mutations in tumor suppressor genes, and thus do not constitute ideal drug targets, since it is difficult to develop drugs that restore the function of a missing or altered protein. It may be possible to achieve this indirectly by inhibiting the activity of a protein that acts downstream of the missing tumor suppressor gene product along a signaling pathway (for example, inhibiting CDK4 activity may correct for the loss of the p161NK4a (C. J. Sherr, Science 274, 1672 (1996)). However, our limited knowledge of mammalian signaling pathways makes this at best a limited and risky approach. An alternative, broader strategy is synthetic lethal screening (reviewed in V. Doye and E. C. Hurt, Trends Genet. 11, 235 (1995). This approach identifies second site mutations that by themselves are not lethal, but in combination with the primary defect, cause lethality. In the setting of anti-cancer drug target identification, the primary defect would be a mutation in a gene conserved from yeast to humans that is frequently inactivated in tumors (e.g., defects in DNA mismatch repair; see Table 1). Gene products with mutations that specifically kill cells with the primary defect would constitute putative “secondary drug targets” (that is, secondary to the primary defect) whose inactivation in tumors may yield great therapeutic advantage.

In principle, synthetic lethality can result when two mutations have an additive negative effect on a single essential biological pathway, or when the mutations inactivate two different but functionally overlapping pathways. One form of genetic instability shows how synthetic lethality could be applied to cancer therapy. All cells use two pathways to eliminate mistakes made during DNA replication: a 3′->5′ proofreading exonuclease activity in DNA polymerase, which eliminates incorrect bases immediately after they are added to the growing chain (See, E. C. Friedberg, G. C. Walker, W. Siede, DNA Repair and Mutagenesis (American Society for Microbiology Press, Washington D.C., 1995); A. Kornberg and T. Baker, DNA Replication, W. H. Freeman and Co., New York), and the mismatch repair system which eliminates mistakes in the newly replicated DNA that have escaped the proofreading activity. See, R. Kolodner, Genes Dev. 10, 1433 (1996). In budding yeast, cells can survive without one of the pathways, albeit with an increased mutation rate. Eliminating both pathways kills yeast cells, presumably because of an excessively high mutation rate. See, A. Morrison, A. L. Johnson, L. H. Johnston, A. Sugino, EMBO J. 12, 1467 (1993).

A hypothetical drug that inhibited the proofreading activity of DNA polymerases delta or epsilon would specifically kill a yeast cell that lacked the mismatch repair system, but not a normal yeast cell. The overlapping functions of the mismatch repair and proofreading are conserved from yeast to humans, as are the proteins that carry them out. Therefore the anti-proofreading drug may be effective in killing tumors with defects in mismatch repair but not in normal proliferating cells.

Synthetic lethality can be detected in two ways: candidate crosses and genome-wide screening. The first method uses prior knowledge to make and test predictions about which combination of mutations will kill cells. This method is applicable to any organism in which mutations can be constructed to order, including budding and fission yeasts, nematodes, flies and mice, and was the method used to show the synthetic lethality of defects in proofreading and mismatch repair. The second method is to perform genetic screens for new synthetically lethal mutations. A strain that carries a single mutation is mutagenized and subjected to various screening programs that will reveal new mutations that are synthetically lethal with the original mutation. This approach requires no prior knowledge but depends on the ability to conduct large scale genetic screens and is currently restricted to microorganisms, nematodes and Drosophila. Once synthetically lethal mutations have been identified, cloning of the corresponding wild-type gene allows identification of the mutated protein and assessment of the suitability of the protein as a target for drug discovery. The complete sequence of the budding yeast genome will allow comprehensive and automated screening for synthetic lethality.

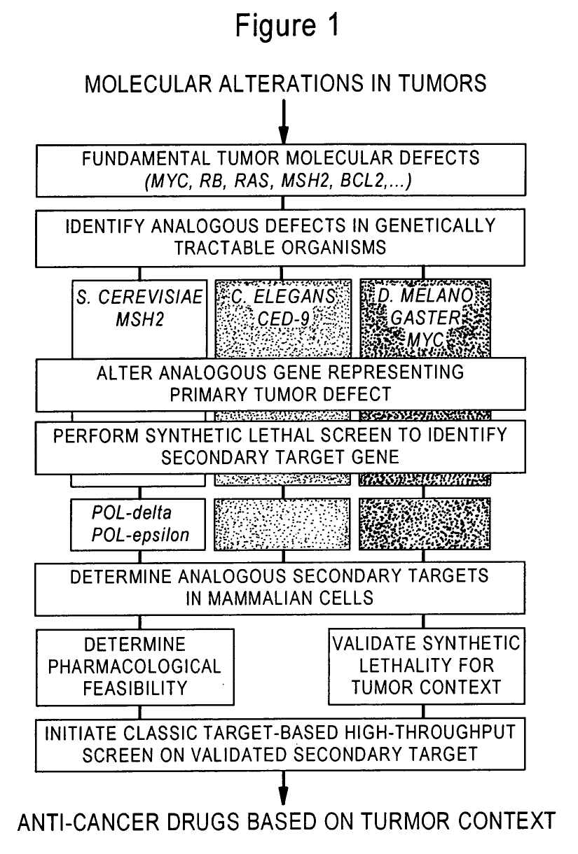

FIG. 1 outlines the steps involved in identifying and using synthetic lethal screens in model genetic organisms to identify secondary targets. Examples of primary tumor defects that can be modeled in facile genetic systems include S. cerevisiae mutants lacking the MSH2 DNA mismatch repair gene, C. elegans mutants defective for the bcl-2 homolog CED-9, and fruit flies overexpressing MYC. Once secondary targets have been identified in the model systems, there are several conditions which must be met before it is reasonable to initiate high-throughput screens for inhibitors of the mammalian homologous of these gene products. It is first necessary to validate that the synthetic lethality also occurs in mammalian cells (both matched pair cell lines and tumor cell lines) in which the primary and secondary targets are inactivated. This will require the use of mammalian inducible gene disruption techniques such as ribozymes, antisense molecules, or dominant-negative strategies. The pharmacological feasibility of each putative drug target must be determined simultaneously, since the secondary targets most amenable to the inhibition by small molecules (for example, enzymes with well-defined substrates) will be the obvious first choices for further analysis. Only after these tests have been completed can the standard high-throughput screens for inhibitors of these validated mammalian secondary targets be initiated.

Accordingly, where target based screens are used, it is possible to exploit the detailed information gathered for several model organisms that are genetically tractable. This approach is well suited to identifying drugs that have a selective killing capacity for the tumor context. The present method allows an alternative to strategies that are based on inhibiting the activities of oncogene products, or attempting to restore the lack of activity resulting from the inactivation of a tumor suppressor gene product. As such genetic approaches allow an alignment of particular molecular defects with “specific” drugs, there is a high probability that the serious side effects associated with many currently used chemotherapeutics will be less problematic.

Many possible targets can be contemplated in view of the disclosure of the present invention. One possible target is an AT-related kinase (ATR). Another can be the cdc53 E3 ubiquitin ligase component. Yet a third is ribonucleotide reductase (RNR). Of these RNR may be the least desirable target because several existing cancer drugs already target this enzyme or other points of nucleotide metabolism. ATR is an attractive target with the availability of certain biological validation results. In particular, test are conducted to determine whether dominant negative ATR is toxic to tumor cells having different mutations of the p16/cdk/Rb/E2F pathway. For example, dnATR is tested in cells with loss of p16, overexpression of cyclins D1 and/or E, or mutant Rb. The effect of dnATR on normal cells can also be examined using transgenic animal models. Cdc53 would also be an attractive target with proof that it is the catalytic component of E3 ligase. The inventors of the present invention have discovered that overexpression of a kinase-inactive ATR protein causes sensitivity to DNA-damaging agents and defects in cell cycle checkpoints. The results of the ATR assay is shown in Figure is 7. Molecular cloning of ATRwt and ATRkd, G2/M checkpoint assay, clonogenic survival assays and DNA synthesis assays are described in Clibly et al. EMBO J. 17,159-169 (1998). The complete disclosure of this reference is incorporated herein by reference.

The invention is illustrated by the following examples, which are not to be construed as limiting in any way.

### EXAMPLE 1

### Yeast Genes with Human Homologues

Yeast-based synthetic lethal screens are put in place to functionally identify and validate new gene targets to kill tumor cells with defects in cell cycle checkpoints and damage response pathways. These newly identified gene targets are then used to develop new cancer chemotherapeutics. Synthetic lethal screening in yeast reagents are prepared and validated. Protein targets are identified for several yeast defects that are analogous to those found in human tumors.

The screening of mutagenized expression libraries in yeast provides numerous genetically linked lethal matches. Random genetic screening focuses on strains defective for msh2 or mlh1 (mismatch repair), or mec1 (a PIK-related kinase having homology to the ataxia-telangiectasia mutation), and a strain overexpressing CLN2 (a cyclin at G0/S). In addition, over 60 known genes are tested for synthetic lethality with these strains.

Using yeast-based assays, approximately 50 genes have been found of which about 35 have human homologues. Essentially all are known genes. The results of the genes with human homologues are listed below. Synthetic lethal screens are conducted with a variety of primary gene defects, including sic1, madl and Rad51. Several lethal secondary site mutations are identified. Alternatively, a yeast screen is put in place to look for genes that are synthetic lethal with elevated telomerase activity (with such synthetic lethality being independent of telomere shortening).

The Table, below, illustrates some of the secondary site mutations found to be lethal in combination with the indicated primary gene defect (the human homologue is also indicated).

| Yeast | Human homologue |
|---|---|
| DNA mismatch repair screen (mlh1, msh2) | |
| DNA ligase (cdc9) | DNA ligase I |
| DNA polymerase 3 (cdc2) | DNA polymerase |
| DNA polymerase 2 | DNA polymerase |
| Ribonucleotide reductase | Same |
| PIK-related kinase screen (mec1) | |
| Thymidylate kinase (cdc8) | Same |
| Thymidylate synthase (cdc21) | Same |
| Ribonucleotide reductase | Same |
| Serine hydroxymethyl transferase | Same |
| DNA repair endonuclease (rad27) | FEN-1 |
| DNA primase | Same |
| DNA polymerase alpha | Same |
| MBP1 transcriptional activator | No. (E2F like) |
| Slm1 | |
| Cyclin overexpression screen (CLN2) | |
| PIK-related kinase (mec1) | AT-related |
| E2 ubiquitin carrier protein (cdc34) | 2 isozymes |
| E3 ubiquitin ligase (cdc53) | 5 isozymes |
| E3 ubiquitin ligase (skp1) | 2 isozymes |
| Protein phosphatase (cdc14) | 2 isozymes |
| Nuclear pore protein (NUP170) | NUP155 |
| CDK inhibitor screen (Sic1) | |
| M phase kinases (cdc15, cdc5) | PLK, PRK |
| Protein phosphatase (cdc14) | 2 isozymes |
| Proteolysis components (cdc16, cdc23, hct1) | APC complex |
| Nuclear pore protein (NUP170) | NUP155 |
| Rad51/52 screen | |
| DNA ligase (cdc9) | DNA ligase I |
| DNA repair endonuclease (rad27) | FEN-1 |
| DNA replication/repair (cdc44, pol130) | PCNA complex |
| Mitotic apparatus screen (mad2) | |
| Microtubule motors (kar3, cin8, kip1) | Kinesin |
| Centromere binding proteins (cbf1, ctf19) | Yes |
| Cyclin clb3 | Cyclin B |
| Mitotic apparatus screen (mad1) | |
| Tubulin alpha and beta | Same |
| Microtubule motor (cin8) | Kinesin |
| Telomerase screen (TLC1) | |

### EXAMPLE 2

### Specific Results of Genes with Human Homologues

| | |
|---|---|
| DNA mismatch repair screen | 3 DNA modifying enzymes |
| (mIh1, msh2) | Ribonucleotide reductase |
| PIK-related kinase screen | 4 nucleic acid pathway enzymes |
| (mec1) | 3 DNA modifying enzymes |
| Cyclin overexpression screen | 3 components of ubiquitination |
| (CLN2) | 1 Protein phosphatase |
| | 1 Nuclear pore protein |
| | 1 PIK-related kinase |
| | (ATR, Ataxia-Telangiectasia Related) |
| CDK inhibitor screen | 3 Proteolysis components of APC complex |
| (Sic1) | 2 M phase kinases |
| | 1 Protein phosphatase |
| | 1 Nuclear pore protein |
| Mitotic apparatus screen | 3 Kinesins |
| (mad ½) | 2 Centromere binding proteins |
| | Cyclin B |
| | Tubulin alpha and beta |

The most advanced target that has been characterized in mammalian cell assays is the protein kinase ATR (ataxia-telangiectasia related). This gene was identified in yeast as being synthetic lethal with elevated cyclin expression. ATR belongs to the family of phosphoinositide-related kinases, which share homology to lipid kinases but are actually protein kinases that are devoid of lipid kinase activity. Like other members of this family, ATR is a very large protein of 2644 amino acids. Beyond the 300 amino acid kinase domain, little is known of the biochemical activities of ATR other than this protein detects damaged DNA and initiates a signal pathway for repair. In mammalian cell assays, transfection of a kinase-defective form of ATR is inhibitory to tumor cell growth and sensitizes immortalized cells to an alkylating agent. Transgenic mice expressing the kinase defective form of ATR in T-cells have normal thymus development and T-cell functions, suggesting that the ATR mutant is not toxic to a normal cell. When the identical mutation in kinase defective ATR is placed into yeast Mec1 (the homologue of ATR), the mutant mec1 is synthetic lethal with cyclin overexpression.

### EXAMPLE 3

### mec 1-Synthetic Lethal Screen (140,000 Colonies were Tested)

| Secondary | | Type of | Number of | | Homolog/ | |
|---|---|---|---|---|---|---|
| Target | Status | Screen | Alleles | Essential? | Analog | Function |
| RNR1 | new | genetic | 24 | Yes | R1 | Large subunit |
| | | | | | | of RNR |
| RNR2 | new | genetic | 2 | Yes | R2 | Small subunit |
| | | | | | | of RNR |
| RNR4 | new | genetic | 1 | Yes | R2 | Small subunit |
| | | | | | | of RNR |
| CDC8 | old | genetic/ | 2 | Yes | Thymidylate | DTMP −> dTDP |
| | | literature | | | kinase | |
| CDC21 | new | genetic/ | 4 | Yes | Thymidylate | DUMP −> dTMP |
| | | crosses | | | synthase | |
| SHM2 | new | genetic | 10 | No | SHMT | generate DCD21 |
| | | | | | | cofactor |
| PRII | new | genetic | 1 | Yes | hp48 | DNA primase subunit |
| CDC17 | new | genetic | 1 | Yes | Pol alpha | DNA polymerase |
| | | | | | | alpha subunit |
| RAD27 | old | crosses | 1 | No | FEN-1 | endonuclease |
| | | | | | | involved in |
| | | | | | | replication and |
| | | | | | | repair |
| MBP1 | old | genetic | 6 | No | E2F | G1-S transcription |
| | | | | | | factor, controls |
| | | | | | | cdc8/cdc21/RNR2 |
| SLM1 | old | genetic | 1 | No | | |
| 141/154 | new | genetic | 2 | | | |
| (SLM2) | | | | | | |
| 87 (SLM3) | new | genetic | 1 | | | |
| 240 (SLM4) | new | genetic | 1 | | | |

### EXAMPLE 4

### Synthetic Lethal Screening Based on Complementing Plasmid Loss

A yeast strain is constructed which bears a mutated copy of the yeast genomic ade2-101, ade3, and ura3 and a mutated copy of an analyzed gene. Additionally, the yeast strain harbors a centromere-containing plasmid that harbors the wild copy of the yeast URA43 and ADE2 and a wild copy of the analyzed gene (plasmid A). The yeast strains harboring plasmid A grow into red colonies. The loss of plasmid A from the yeast strain corresponds to the loss of the color red which results in production of white colonies.

The yeast strain described above is mutagenized with either EMS or UV with 10-30% survival rate. Induced mutagenesis is screened by a relative increase in forward can 1 locus mutation frequency. Then the titer of the mutagenized cells are measured by plating 500 colony forming units per 120 mm Petri dish on an agar-based media containing necessary salts, vitamins, dextrose, uracil, leucine and adenine. Mutagenized cells form colonies on this media and lose plasmid A since the media contains all nutrients for which the yeast strain is auxotrophic. The colonies growing on this media contain white sectors because the cells that lose the plasmid produce white colonies.

Mutant colonies that are synthetically lethal with the analyzed gene require the wild type copy of the analyzed gene of interest, which is harbored by plasmid A, to be present in the cell for viability. Therefore, viable mutant colonies must necessarily contain plasmid A to grow. The mutant colonies that retain the plasmid appear to be homogeneously red in color.

The red colonies thus obtained are re-screened on the same media for the phenotype of non-sectoring. Then the cells are tested for their sensitivity to grow on plates containing 5-fluoroorotic acid. This test allows for the determination of whether or not another marker on the plasmid (URA3) is retained within the cells. Since the likelihood that the two markers being retained independent of the plasmid is small, it is concluded that the whole plasmid is retained as an episome.

After establishing that the cells of a particular isolate retain the plasmid, these cells are transformed with another plasmid, containing another copy of the analyzed gene (plasmid B). Those isolates which showed a destablization of the analyzed gene upon introduction of plasmid B are selected for further analysis. Destablization of the analyzed gene in these cells is an indication that plasmid A is retained in these cells because the cells relied on the wild type copy of the analyzed gene to survive.

The cells containing plasmid A, as determined above, are then crossed with the wild type strain of the opposite mating type. If plasmid A becomes destabilized in the resulting progeny, it is concluded that the synthetic lethal mutation in the isolate is recessive and, therefore, can be cloned by complementation. If plasmid A does not become destabilized after crossing with the wild type strain, the mutation is dominant and is omitted from the successive analysis.

The isolates that contain recessive synthetic lethal mutation, crossed with the wild type strain, are sporulated and dissected and the spore tetrads are screened for the appropriate single mutation pattern of segregation of the synthetic lethal phenotype. The mutants that show appropriate pattern of segregation are then cloned by complementation.

### EXAMPLE 5

### Synthetic Lethal Screening Based on Inducible Expression of the Analyzed Gene

A yeast strain is constructed which bears a wild type copy of the analyzed gene under the control of an inducible promoter from the GAL1 gene. Then the strain is mutagenized to yeild a 10-30% survival rate with either EMS or UV. The induced mutagenesis is determined by a relative increase in forward can 1 locus mutation frequency. The mutagenized cell titer is then determined by plating 500 colony forming units per 120 mm Petri dish on an agar-based media containing necessary salts, vitamins and galactose and sucrose. In two to three days, when the colonies have grown, the Petri dishes containing mutagenized colonies are replica-plated on two similar plates, having-either dextrose, or galactose/sucrose as a carbon source. Colonies containing mutations synthetically lethal with the analyzed gene will grow on galactose but will not grow on dextrose-containing media. Such isolates are re-screened for the absence of growth on dextrose-containing media.

The colonies containing a synthetic lethal mutation are then transformed with a plasmid bearing a copy of the gene being analyzed. If the colonies indeed contain a synthetic lethal mutation with the analyzed gene, then introduction of such plasmid into the cells should allow transform cells to grow on dextrose-containing media. The transformed cells are then crossed with the wild type strain of the opposite mating type. If the hybrid strain grow on glucose, it is concluded that the synthetic lethal mutation in the isolate is recessive and, therefore, can be cloned by complementation. If the hybrid strain cannot grow on glucose, the mutation is dominant and it is omitted from the successive analysis.

The hybrids that contain recessive synthetic lethal mutations are crossed with the wild type strain, sporulated and dissected, and the spore tetrads are checked for the appropriate single mutation pattern of segregation of the synthetic lethal phenotype. The mutants that show such pattern of segregation are cloned by complementation.

### EXAMPLE 6

### The Telomerase-Dead Screen

The mutants, which will survive only in the absence of telomerase function in yeast, are screened as follows. The yeast strain used contain the EST1, EST2 and TEL1 genes under the inducible promoter from the GAL1 gene on circular centromeric plasmids. The strain is mutagenized to yeild a 10-30% survival rate with either EMS or UV. The induced mutagenesis is determined by a relative increase in forward can 1 locus mutation frequency. Then the mutagenized cell titer is determined by plating 500 colony forming units of mutagenized cells per 120 mm Petri dish, on an agar-based media containing necessary salts, vitamins and dextrose. In two to three days, when the colonies have grown, the Petri dishes containing mutagenized colonies are replica-plated on three similar plates, having dextrose, galactose or glycerol as a carbon source. The colonies that did not grow on galactose-containing media, but grew on dextrose and glycerol-containing media are selected. Colonies formed by respiration-deficient cells will not grow on glycerol and thus are omitted from further analysis. The isolates are re-screened for the absence of growth on galactose-containing media.

The successful isolates are then allowed to lose the TEL1-containing plasmid. This should allow the mutant cells to grow on galactose-containing media. Then the successful isolates are crossed with the wild type strain of the opposite mating type. If the hybrid strain grow on galactose, it is concluded that the synthetic lethal mutation in the isolate is recessive and, therefore, can be cloned by complementation. If the hybrid strain cannot grow on glucose, the mutation is dominant and it is omitted from the successive analysis. The hybrids that contain recessive synthetic lethal mutations are crossed with the wild type strain sporulated and dissected and the spore tetrads are checked for the appropriate single mutation pattern of segregation of the synthetic lethal phenotype. The mutants that show such pattern of segregation are cloned by complementation.

### EXAMPLE 7

### Assay for the Drosophila Screen

This screen uses the rough eye phenotype caused by overexpression of the myc oncogene. Enhancers make the phenotype worse whereas suppressors alleviate the phenotype. In the view of the inventors the enhancers are the conceptual equivalent of synthetic lethal.

Modifiers of the “rough eye” phenotype caused by ectopic expression of dmyc and dmax in the eye.

A. Enhancers:

A1. By overexpression:
PI3 kinase (wild type)
E2F+DP1
cyclin A
cyclin E
   B. Suppressors:
B 1. By overexpression:
string (Drosophila cdc25)
PI3 kinase (dominant negative)
   RBF
da capo (a cdk-inhibitor)
p35 (a viral inhibitor of apoptosis)
   B2. By Mutation:
DCP-1 (a caspase)
Df(3L)H99 (a deficiency removing the apoptosis-inducing genes grim, hid, reaper) pitchoune (a homolog of MrDb)

### EXAMPLE 8

### Genetic Screen for Dominant Modifiers of a dmyc Overexpression Phenotype

Similar screens have been carried out to identify genes interacting with members of the Ras signal transduction pathway; e.g. Neufeld et al. 1998, Genetics 148, 1-10. Male flies of the genotype “w[−]; iso 2,3” (i.e., carrying isogenic 2nd and 3rd chromosomes) are mutagenized with ethyl methanesulfonate (EMS) as described by T. Grigliatti (in Drosophila: A practical approach. D. B. Roberts (ed.). IRL Press; Oxford, Wash. (1986). pp. 39-58). They are then mated in mass with virgin females of the genotype “w[−]; GMR-GAL4 UAS-dmyc[132]UAS-dmax[14]/CyO; UAS-dmyc[13] UAS-dmyc[42]” (called “GMM” in short). GMM flies express dmax and dmyc specifically in the differentiating part of the eye imaginal discs (posterior to the morphogenetic furrow), causing them to have an aberrant eye morphology as adults. Male Cy[+] offspring from the above mating is analysed visually for their eye morphology, using a dissecting microscope. Flies derived from non-mutagenized fathers will have a moderately disturbed eye morphology. Flies with more severe or less severe eye defects have potentially acquired mutations in genes interacting with dmyc. These mutations are termed “suppressors” and “enhancers”, respectively, and further characterized.

First it is established whether the identified enhancer or modifier mutation is genetically inheritable. Second, using appropriate tester lines, the chromosomal linkage of the mutation is determined. Third, the mutation is balanced and established as a stock. In further steps, the identity of the different mutated genes will be addressed and their functional interaction with dmyc characterized.

### EXAMPLE 9

### ATR Assay

The ATR protein or a fragment of the ATR protein containing the kinase domain is expressed in recombinant form (such as in insect cells). Preferably, the construct used for expression includes a heterologous tag (such as His, FLAG, middle-T, and the like) placed on the protein to enable rapid purification. The purified ATR kinase is then incubated in an appropriate buffer solution containing gamma-³³P or gamma-³²P labeled ATP and protein substrate. Substrates of ATR include, but are not limited to, p53 protein, myelin basic protein and the like. Other appropriate substrates are well known to those of ordinary skill in the art. The ensuing reaction produces radiolabeled substrate protein, which can be isolated by several methods including acid precipitation onto filter paper. If the substrate has a tag such as GST, then radiolabeled substrate can be captured on flashplates (New England Nuclear) or SPA beads (Amersham). Non-radioactive methods of detection are also possible once antibodies are made to recognize the phosphorylated reaction product.

Hence, the above-described elements comprise suitable components of a screen that can be used to identify compounds that inhibit the activity of ATR. The compounds revealed by the screen to exhibit inhibitory activity are considered potential drugs for the treatment of one or more types of cancer.

It should be evident to one of ordinary skill in the art that other embodiments of the invention are possible given the detailed descriptions provided herein. It should be equally evident that the present invention is not limited solely to the specific embodiments provided herewith but by the claims that follow.

1. A method of identifying a secondary drug target comprising:
(a) providing a cell having a genome, including at least one primary gene defect;
(b) effecting one or more mutations in said genome of said cell, at one or more secondary sites;
(c) selecting at least one secondary site mutation that proves lethal to said cell; and
(d) determining the gene product of said lethal secondary site to provide a secondary drug target.

2. The method of claim 1 in which said primary gene defect is found in or associated with a human tumor.

3. The method of claim 1 in which said primary gene defect is analogous or homologous to a defect found in or associated with a human tumor.

4. The method of claim 1 in which said primary gene defect results in the alteration, loss, inhibition, enhancement, or gain of a function.

5. The method of claim 4 in which said function includes the suppression of tumor growth, DNA damage checkpoint, DNA mismatch repair, nucleotide excision repair, O6-methylguanine reversal, double-strand break repair, DNA helicase function, signaling, cell cycle control, or apoptosis.

6. The method of claim 5 in which said signaling function includes signal transduction, tissue growth factor signaling, autocrine loop signaling, or paracrine loop signaling.

7. The method of claim 1 in which said primary gene defect includes a defect in the gene coding for p16, p53, ATM, MSH2, MLH1, XP-A, XP-B, MGMT, BRCA2, BRCA1, BLM, RAS, NF1, MYC, PTH, Cyclin D, Cyclin E, p27kip1, Rb, or BCL-2.

8. The method of claim 1 in which said primary gene defect includes a defect in the gene coding for RAD9Sc, rad1+Sp, MEC1Sc, TEL1Se, rad3+Sp, mei-41Dm, MSH2Sc, MLH1Sc, RAD14Sc, RAD25Sc, MGT1Sc, RAD51Sc, RAD54Sc, SGS1Sc, rgh1+Sp, dRASDm, RASCe, RAS1 Sc, RAS2Sc, let-60Ce, IRA1Sc, IRA2Sc, dMycDm, patchedDm, CLN1Sc, CLN2Sc, Cyclin DDm, Cyclin EDm, SIC1Sc, RbfDm, or ced-9Ce.

9. The method of claim 1 in which said primary gene defect includes a defect in a gene coding for CLN2.

10. The method of claim 1 in which said secondary site mutation is effected within a gene selected from the group consisting of cdc9, cdc2, a gene encoding a gene product exhibiting polymerase 6 exonuclease function, a gene encoding a gene product exhibiting polymerase δ exonuclease function, a gene encoding a ribonucleotide reductase, mec1, rad53 like gene, cdc53, cdc34, cdc14, cdc 15, a gene encoding NUP170, dbf2, a gene encoding CLN2, rad3, rad9, rad27, cdc8, a gene encoding Mlu1-box binding factor, slm1, a gene encoding MBF, a gene encoding PCNA, or a gene encoding a replication fork protein.

11. The method of claim 1 in which said secondary site mutation is effected within a gene coding for a gene product selected from PIK-related kinase (mec 1), E2 ubiquitin carrier protein (cdc34), E3 ubiquitin ligase (cdc53), ubiquitin ligase (skp1), protein phosphatase (cdc 14) and nuclear pore protein (NUP170).

12. The method of claim 1 in which said secondary site mutation is effected within a gene having a mammalian analog or homolog.

13. The method of claim 12 in which said homologous mammalian gene is selected from the group consisting of a gene encoding a DNA ligase I, a gene encoding a DNA polymerase, a gene encoding a ribonucleotide reductase, a gene encoding a FEN-1, a gene encoding Cyclin D, a gene encoding Cyclin E, an AT-related gene, a gene encoding NUP155, or a gene encoding an isozyme.

14. The method of claim 1 in which said primary gene defect includes a defect in a gene coding for CLN2 and said secondary site mutation is effected within a gene coding for a gene product selected from PIK-related kinase (mec1), E2 ubiquitin carrier protein (cdc34), E3 ubiquitin ligase (cdc53), ubiquitin ligase (skp1), protein phosphatase (cdc14) and nuclear pore protein (NUP170).

15. The method of claim 1 which further comprises using said secondary drug target to screen for a drug or drug candidate.

16. The method of claim 15 in which said drug or drug candidate interacts with, binds to, or inhibits a gene product selected from the group consisting of DNA ligase, DNA polymerase, polymerase δ exonuclease, polymerase E exonuclease, ribonucleotide reductase, a subunit of transcriptional activator, a transcription factor, PCNA, a replication fork protein, PIK-related kinase, recombinase, E3 ubiquitin ligase, E2 ubiquitin carrier protein, a protein tyrosine phosphatase, a nuclear pore protein, cyclin, DNA repair exonuclease, thymidylate kinase, gene product of slm1, ribonucleotide reductase, or a transcriptional activator.

17. The method of claim 15 in which said drug or drug candidate inhibits the growth of a human tumor.

18. A method of rational antitumor drug design comprising: (i) providing a genetically tractable organism harboring an altered gene that is analogous or homologous to a primary tumor defect, (ii) performing a synthetic lethal screen to identify a secondary target gene, (iii) determining an analogous or homologous secondary target in mammalian cells, and (iv) using said analogous or homologous secondary target to screen for a drug or drug candidate having antitumor activity.

19. The method of claim 18 in which said drug or drug candidate comprises a small molecule.

20. The method of claim 17 which further comprises validating the synthetic lethality of said analogous or homologous secondary target in a mammalian tumor cell relative to a mammalian non-tumor cell.

21-22. (canceled)

23. The method of claim 1 in which said primary gene defect includes a defect in a gene coding for PIK-related kinase (mec 1).

24. The method of claim 1 in which said secondary site mutation is effected within a gene selected from the group consisting of RNR1, RNR2, RNR4, CDC8, CDC21, SHM2, PR11, CDC17, MBP1, SLM1, SLM2, SLM3, and SLM4.

25. The method of claim 1 in which said primary gene defect includes a defect in a gene coding for PIK-related kinase (mec1), and said secondary site mutation is effected within a gene selected from the group consisting of RNR1, RNR2, RNR4, CDC8, CDC21, SHM2, PR11, CDC17, MBP1, SLM1, SLM2, SLM3, and SLM4.

26-46. (canceled)

47. A method of identifying a secondary drug target comprising:
(a) providing a cell having a genome, including at least one primary gene defect; wherein said cell is selected from the group consisting of Drosophila melanogaster, C. elegans, Saccharomyces cerevisiae, and Schizzosaccharomyces pombe cells;
(b) effecting one or more mutations in said genome of said cell;
(c) selecting at least one secondary site mutation that results in a gene or protein that is non-functional and which proves lethal to said cell; and
(d) determining the identity of said gene or protein that is non-functional to provide a secondary drug target.

48. The method of claim 47, wherein the cell is a Drosophila melanogaster cell and the primary defect is in the Drosophila analog or homolog of the human Rb gene, Cyclin D gene, Cyclin E gene, MYC gene, PTH gene, or ATM gene.

49. The method of claim 47, wherein the cell is a Scizzosaccharomyces pombe cell and the primary defect is in the Scizzosaccharomyces anolog or homolog of the human p53 gene, ATM gene, or BLM gene.

50. The method of claim 47, wherein the cell is a Saccharomyces cerevisiae and the primary defect is in the Saccharomyces anolog or homolog of the human p53 gene, ATM gene, MLSH2 gene, MLH1 gene, XP-A gene, XP-B gene, MGMT gene, RAS gene, BLM gene, NF1 gene, Cyclin D, Cyclin E, or p27 gene.

51. The method of claim 47 wherein the cell is a Caenorhabditis elegans cell and the primary defect is in the Caenorhabdilis analog or homolog of the human BCL-2 gene.
